(19) United States
(12) Patent Application Publication
(10) Pub. No.: US 2026/0167588 A1
(43) Pub. Date: Jun. 18, 2026

(54) APPARATUS AND METHOD FOR PROPOGATION REACTION OF HYDROCARBONS

(71) Applicant: PlasMerica, LLC, Miami, OK (US)

(72) Inventors: Dennis Keith Manning, Miami, OK (US); John C. Frisbie, Miami, OK (US); Jerrold J. Pellizzon, Miami, OK (US)

(21) Appl. No.: 19/129,146

(22) PCT Filed: Nov. 13, 2023
(86) PCT No.: PCT/US2023/079534

§ 371 Date: May 12, 2025

Related U.S. Application Data
(60) Provisional application No. 63/383,669, filed on Nov. 14, 2022.

(51) Int. Cl. C07C 2/80; B01J 19/08; C10G 50/00

(52) U.S. Cl. CPC C07C 2/80; B01J 19/088; C10G 50/00; B01J 2219/0809; B01J 2219/0815; B01J 2219/083; B01J 2219/0835; B01J 2219/0869; B01J 2219/0875; B01J 2219/0896; B01J 2219/1925; B01J 2219/1943; B01J 2219/24; C10G 2300/1025

(57) ABSTRACT

A reaction vessel may include a vessel wall forming an internal volume comprising a fluid space. A reaction vessel may include at least one grounded surface or electrode. A reaction vessel may include at least one dielectric insulator, the at least one dielectric insulator in contact with the at least one grounded surface or electrode. A reaction vessel may include at least one high voltage electrode disposed within the reaction vessel, wherein a first portion of the at least one high voltage electrode is in contact with the at least one dielectric insulator, and wherein a second portion of the at least one high voltage electrode is in contact with the fluid space.

### BACKGROUND

The field relates to apparatuses and methods for hydrocarbon propagation.

### SUMMARY

In some aspects, the techniques described herein relate to a reaction vessel including: a vessel wall forming an internal volume including a fluid space; at least one grounded surface or electrode; at least one dielectric insulator, the at least one dielectric insulator in contact with the at least one grounded surface or electrode; and at least one high voltage electrode disposed within the reaction vessel, wherein a first portion of the at least one high voltage electrode is in contact with the at least one dielectric insulator, and wherein a second portion of the at least one high voltage electrode is in contact with the fluid space.

In some aspects, the techniques described herein relate to a reaction vessel wherein the vessel wall includes the at least one grounded surface or electrode.

In some aspects, the techniques described herein relate to a reaction vessel, wherein the at least one grounded surface or electrode is formed on the vessel wall.

In some aspects, the techniques described herein relate to a reaction vessel, wherein the at least on high voltage electrode is configured to generate an electric field within the reaction vessel.

In some aspects, the techniques described herein relate to a device, wherein the reaction vessel further includes a power supplier electrically connected to the at least one high voltage electrode, the power supplier configured to supply power to the at least one high voltage electrode in a form of an application voltage between 30,000 VAC and 50,000 VAC.

In some aspects, the techniques described herein relate to a reaction vessel, wherein the at least one dielectric insulator is disposed on an inner surface of the at least one grounded surface or electrode.

In some aspects, the techniques described herein relate to a reaction vessel, wherein the reaction vessel is cylindrical.

In some aspects, the techniques described herein relate to a reaction vessel, wherein the at least one high voltage electrode and the at least one dielectric insulator are disposed concentrically or coaxially within the reaction vessel.

In some aspects, the techniques described herein relate to a reaction vessel, wherein the reaction vessel is a rectangular prism and wherein the at least one high voltage electrode is a plate electrode.

In some aspects, the techniques described herein relate to a reaction vessel, including a plurality of high voltage electrodes and a plurality of dielectric insulators arranged parallel to each other within the reaction vessel.

In some aspects, the techniques described herein relate to a reaction vessel, wherein the energized surface area to volume ratio is between 0 and 1.

In some aspects, the techniques described herein relate to a reaction vessel, wherein the at least one high voltage electrode includes a mesh.

In some aspects, the techniques described herein relate to a reaction vessel, wherein the at least one high voltage electrode is configured to allow a fluid to flow through the mesh of the at least one high voltage electrode.

In some aspects, the techniques described herein relate to a method for propagating hydrogens, the method including: providing a feed fluid to a reaction vessel via a fluid inlet, wherein the reaction vessel includes: a grounded surface or electrode; a dielectric insulator in contact with the grounded surface or electrode; a high voltage electrode in contact with the dielectric insulator and a fluid space of the reaction vessel; generating an electric field within the fluid space of the reaction vessel; reacting one or more components of the feed fluid in the electric field; and extracting one or more components of interest from the reaction vessel, via a fluid outlet of the reaction vessel.

In some aspects, the techniques described herein relate to a method, wherein generating an electric field includes creating a plasma in the fluid space near the surface of the high voltage electrode.

In some aspects, the techniques described herein relate to a method, wherein the high voltage electrode at between 30,000 VAC and 50,000 VAC.

In some aspects, the techniques described herein relate to a method, further including adjusting a retention time of the feed fluid within the reaction vessel such that feed fluid remains in the reaction vessel for between 30 seconds and 5 minutes.

In some aspects, the techniques described herein relate to a method, wherein the feed fluid is compressed natural gas (CNG).

In some aspects, the techniques described herein relate to a method, wherein the feed fluid includes one or more hydrocarbon components, and wherein reacting the one or more hydrocarbon components of the feed fluid includes propagating the hydrocarbon components into larger hydrocarbon chains.

In some aspects, the techniques described herein relate to a method, wherein extracting the component of interest includes extracting hydrocarbon chains from the reaction vessel.

### BRIEF DESCRIPTION OF THE DRAWINGS

The detailed description is set forth with reference to the accompanying figures. The use of the same numbers in different figures indicates similar or identical items.

For this discussion, the devices and systems illustrated in the figures are shown as having multiple components. Various implementations of devices and/or systems, as described herein, may include fewer components, and remain within the scope of the disclosure. Alternatively, other implementations of devices and/or systems may include additional components, or various combinations of the described components, and remain within the scope of the disclosure.

The foregoing and other features of the disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A is a cross section of an exemplary reaction vessel with the components in an annular arrangement.

FIG. 1B is a top view of a reaction vessel as portrayed in FIG. 1A.

FIG. 2A is a cross section of an exemplary reaction vessel.

FIG. 2B is a top view of the reaction vessel of FIG. 2A.

FIG. 3A is a cross section of an exemplary reaction vessel.

FIG. 3B is a top view of the reaction vessel of FIG. 3A.

FIG. 4 is a cross section of an exemplary reaction vessel with the components in a parallel plate arrangement.

### DETAILED DESCRIPTION

In the following detailed description, reference is made to the accompanying drawings, which form a part hereof. In the drawings, similar symbols typically identify similar components, unless context dictates otherwise. Thus, in some embodiments, part numbers may be used for similar components in multiple figures, or part numbers may vary from figure to figure. The illustrative embodiments described herein are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

The following detailed description is directed to certain specific embodiments of the development. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Furthermore, embodiments of the development may include several novel features, no single one of which is solely responsible for its desirable attributes or which is essential to practicing the invention described herein.

Reactors described herein for hydrocarbon propagation in gas ionization and/or plasma phase reactions can comprise one or more high voltage electrodes, a solid phase dielectric, the gas to be ionized, and a ground reference. In some embodiments, the components are arranged such that the dielectric and gap(s) are located between the high voltage electrode and the ground reference. In some embodiments, the dielectric is in contact with the gas to be ionized on one side and the high voltage electrode on the other side of the dielectric and the gas is located between the dielectric and ground. These allow a gradient potential to be created across the gas gap between the energized dielectric surface and the ground reference.

The arrangement of the components within the reactor affects the amount or extent of the propagation reaction. The role and placement of the dielectric insulator in relation to the high voltage electrode and ground reference has previously not been fully appreciated. Embodiments described herein have discovered advantageous arrangements of electrode and dielectric configurations, as well as electrical and physical parameters to affect and improve reaction effectiveness and reaction extent. For example, changing the ratio of the energized surface area of the dielectric insulators to the free gas volume affects reaction effectiveness and propagation extent, energy requirements, and other reaction parameters. This ratio can be referred to herein as the energized surface area to volume ratio. Thus, adjusting dielectric and electrode configurations within reactor vessels may lead to greater reaction effectiveness. Additionally, different arrangements of the electrodes and dielectric(s) may have additional benefits, for example, in reducing the quantity of materials used, minimizing reactor vessel size, improved reaction efficiency, improved product yields, and reduced costs.

Using systems and methods described herein, small chain hydrocarbons can be converted into longer chain hydrocarbons. Some hydrocarbon related processes, such as hydrocarbon production or other processes can generate a waste stream of methane, or of low molecular weight hydrocarbons. By introducing the waste stream into reactors as described herein can convert otherwise waste streams into useable hydrocarbon products. In some embodiments, natural gas or other naturally occurring hydrocarbons can be processed and converted into higher molecular weight hydrocarbons without the need for traditional chemical reactions, such as the refining and breaking down of long-chain hydrocarbons, which can result in undesirable byproducts and/or environmental impacts.

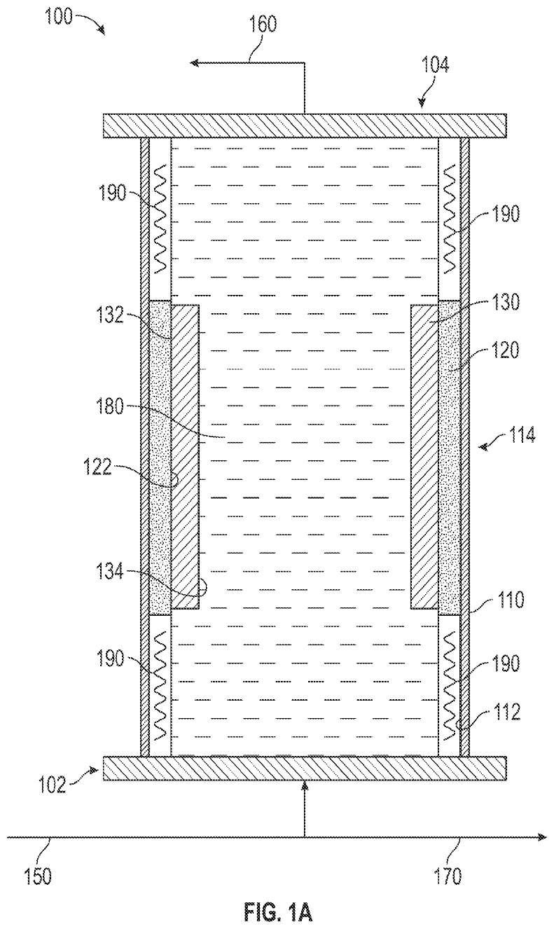

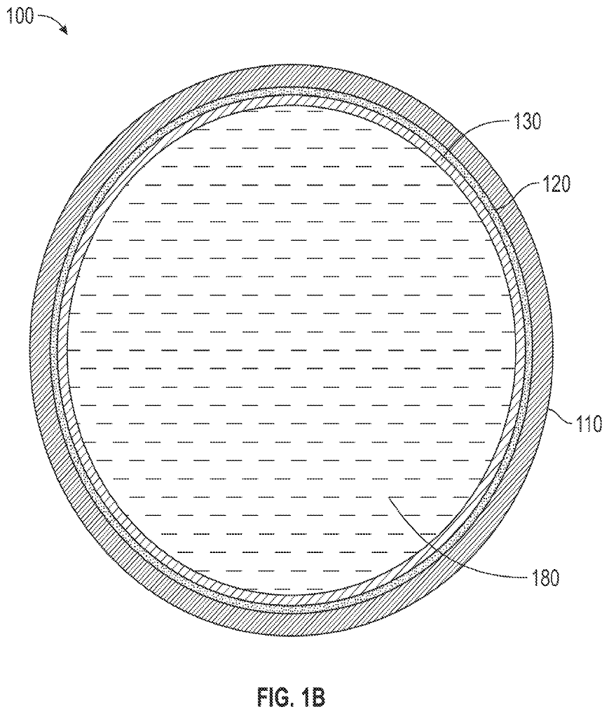

FIG. 1A is a cross section of a reaction vessel 100 with the components in an annular arrangement. The reaction vessel 100 includes a grounded surface or electrode 110, a dielectric insulator 120, an internal high voltage electrode 130, a power supply/controller 140, a gas inlet 150, a gas outlet 160, and a liquid outlet 170.

As shown in FIG. 1A, the grounded surface or electrode 110 is the outer wall of the reaction vessel. In some embodiments, the grounded surface or electrode 110 may be formed on or contact an inner surface 112 of the reaction vessel wall. In some embodiments, the grounded surface or electrode 100 may cover the entire inner surface 112 of the reaction vessel wall 114. In some embodiments, the grounded surface or electrode 110 may cover a portion of the inner surface 112 of the reaction vessel wall 114.

As shown in FIG. 1A, the dielectric insulator 120 is placed in such a manner as to contact the grounded surface or electrode 110. In some embodiments that dielectric insulator 120 may cover the entire grounded surface or electrode 110. In some embodiments, the dielectric insulator 120 may only cover a portion of the grounded surface or electrode 110. In some embodiments, the dielectric insulator 120 may incorporate corrugated ends 190 to reduce voltage tracking along the inner surface 112 of the vessel 100. In some embodiments, the dielectric insulator 120 is corrugated along the length or a substantial portion thereof. Using a corrugated dielectric insulator can increase the dielectric surface area within the reaction vessel 100 compared to a non-corrugated dielectric insulator.

The dielectric insulator 120 acts as an electric insulator for the electric field generated within the reaction vessel 100. The dielectric insulator 120 can act as a barrier between the high voltage electrode 130 and the grounded surface or electrode 100. This arrangement is advantageous to enabling, enhancing, and/or increasing propagation reactions of hydrocarbons.

The dielectric insulator 120 may be composed of various materials. In some embodiments, the dielectric insulator 120 may be composed of quartz, glass, ceramics, polymers, or other suitable dielectrics. In some embodiments the dielectric insulator 120 may be impregnated and/or coated with catalytic materials to promote reaction kinetics. In some embodiments, the catalytic materials may comprise iron, magnesium, nickel, cobalt, thorium, ruthenium, or other suitable catalytic material. In some embodiments, the catalytic materials may comprise a mixture or combination of the materials described above or other suitable materials.

As shown in FIG. 1A, the high voltage electrode 130 is located within the reaction vessel 100 proximate to the dielectric insulator 120. The high voltage electrode 130 contacts the dielectric insulator 120. A first side 132 of the high voltage electrode 130 is placed along and in contact the dielectric insulator's inner surface 122 with a second side 134 of the high voltage electrode 130 exposed to the fluid space or internal volume 180 of the reaction vessel 100, where, for example, the high voltage electrode 130 is in contact with the feed hydrocarbons, free gas, reaction products, and/or any combination of the above. In some embodiments, the high voltage electrode 130 may be suspended within the dielectric annulus between the corrugated ends 190 of the dielectric insulator 120 to minimize arcing potential to the grounded surface or electrode 110. In some embodiments, the high voltage electrode 130 may extend the entire length of the dielectric insulator 120. In some embodiments, the high voltage electrode 130 may extend along only a portion of the dielectric insulator 120.

The high voltage electrode 130 may be composed of various materials. In some embodiments, the high voltage electrode 130 may be a mesh. Such embodiments may be advantageous for allowing a gas to flow through the high voltage electrode 130 and to contact the dielectric insulator 120 along the length of the high voltage electrode 130. In some embodiments, the high voltage electrode 130 may be a copper mesh, for example, a copper mesh with a 50% open area. In some embodiments, the copper mesh may have a different open area percentage, e.g., 25%, 75%. In some embodiments, the high voltage electrode 130 may be formed from another material, such as carbon steel, or a nickel-iron alloy. In some embodiments, the high voltage electrode 130 may be formed from a catalytic material. For example, an iron-nickel alloy may be an appropriate catalytic material for a desired reaction, and the high voltage electrode 130 may be an iron-nickel alloy mesh. In some embodiments, the high voltage electrode 130 may be impregnated or coated with catalytic materials. In some embodiments, the catalytic materials may comprise iron, magnesium, nickel, cobalt, thorium, ruthenium, or other suitable catalytic material. In some embodiments, the catalytic materials may comprise a mixture or combination of the materials described above or other suitable materials.

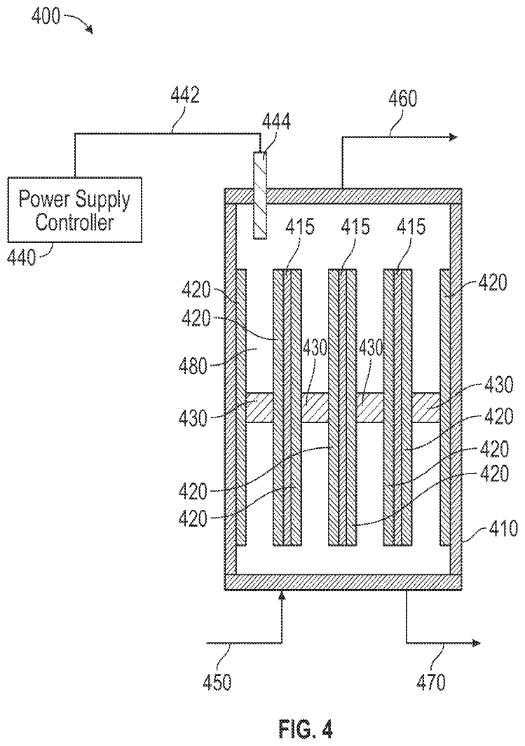

The reaction vessel 100 includes a power supply/controller (for example as seen in FIG. 4. The power supply/controller is configured to supply power to the high voltage electrode 130 to generate an electric field. In some embodiments, the power supply/controller can be coupled to the high voltage electrode 130, for example, via a high voltage cable (for example as seen in FIG. 4). In some embodiments, a high voltage cable 142 can extend between the power supply/controller 140 and the electrode 130 through the gas inlet 150. In some embodiments, the high voltage cable 142 can extend between the power/supply controller 140 and the high voltage electrode 130 through the gas outlet 160. In some embodiments, the vessel 100 can include a high voltage entrance device 144 (as seen in FIG. 4). The high voltage entrance device 144 may be a high voltage insulator entrance bushing.

In some embodiments, power can be supplied to the electrode 130 in the form of an application voltage of between 100 VAC and 100,000 VAC, between 100 VAC and 10,000 VAC, between 500 VAC and 1,500 VAC, about 500 VAC, about 1000 VAC, about 5,000 VAC, about 10,000 VAC, about 45,000 VAC, about 50,000 VAC, about 60,000 VAC or any other suitable application voltage. In some embodiments, the voltage range can advantageously be between 12,000 VAC and 15,000 VAC, or between 35,000 VAC and 60,000 VAC.

In certain embodiments, power can be supplied to the electrode 130 in the form of an application frequency of between 60 hertz to 10,000 hertz, or any other suitable frequency. In some embodiments, the frequency can advantageously be between 1,000 hertz and 1,200 hertz.

As shown in FIG. 1A, the gas inlet 150 is configured to allow a gaseous feed stream to flow into the reaction vessel 100 through the bottom 102 of the reaction vessel 100. In some embodiments, the gas inlet 150 may be configured to allow a gaseous feed stream to flow into the reaction vessel 100 from another portion of the reaction vessel 100. For example, the gas inlet 150 may be situated to allow a gaseous feed stream to flow in the reaction vessel 100 from the top 104 of the reaction vessel 100. In some embodiments, the flow rate may be adjusted to alter retention times of the gas within the reaction vessel 100. The gas inlet 150 includes one or more components which enable controlling the flow rate of a feed stream into the reaction vessel. Controlling the flow rate of the feed stream can control the dwell time or retention time of the feed stream in the reaction vessel 150. In some embodiments, the diameter of the gas inlet 150 may be increased or decreased to alter the flow rate of a gaseous feed stream entering the reaction vessel 100.

As shown in FIG. 1A, the gas outlet 160 is configured to allow gaseous product to exit the reaction vessel 100 through the top 104 of the reaction vessel 100. In some embodiments, the gas outlet 160 may be configured to allow a gaseous product stream to exit the reaction vessel 100 through another portion of the reaction vessel 100. In some embodiments, the diameter of the gas outlet 160 may be increased or decreased to alter the flow rate of a gaseous product stream exiting the reaction vessel 100. In some embodiments, the gas outlet 160 can include one or more components to control the rate of fluid flow out of the reaction vessel 100, and, in conjunction with the gas inlet 150, can control or adjust retention time of fluid in the reaction vessel 100.

As shown in FIG. 1A, the liquid outlet 170 is configured to allow a liquid product stream to exit the reaction vessel 100 through the bottom 102 of the reaction vessel 100. In some embodiments, the liquid outlet 170 may be configured to allow a liquid product stream to exit the reaction vessel 100 through another portion of the reaction vessel 100. In some embodiments, the diameter of the liquid outlet 170 may be increased or decreased to alter the flow rate of a liquid product stream exiting the reactor vessel 100. In some embodiments, a pump may be in fluid communication with the liquid outlet 170 downstream of the reaction vessel 100. In some embodiments, the reaction products may be in the gas phase, and the product stream may be a gas-phase product stream. In some embodiments, the product stream may have a gas-phase and a liquid-phase component.

The reaction vessel 100 can be designed to operate at low, moderate, or high pressures depending on the application demand. In some embodiments, the pressure can be 2 psig. In some embodiments, the pressure may be related to the quantity of propagation reactions taking place. In some embodiments, increasing pressure may force the molecules of the feed gas into closer proximity and increase gas conductivity. This may improve energy transfer through the gas phase. In some embodiments, greater pressures may provide greater mass throughput.

FIG. 1B depicts a top view of the reaction vessel 100 as portrayed in FIG. 1A with a top portion of the vessel 100 removed. FIG. 1B shows the reaction vessel 100 and components configured in an annular arrangement. While an annular reaction vessel can advantageously be employed, other reaction vessel configurations can also be employed in certain embodiments. These configurations may include, but are not limited to: concentric electrodes configurations, multiple concentric electrodes configurations, parallel plate systems, multiple plate configurations, honeycomb configurations, cylindrical, spherical, or any other desired configuration. One of skill in the art would understand, aided by the current disclosure, that different vessel sizes and shapes can be used without departing from the scope of this invention.

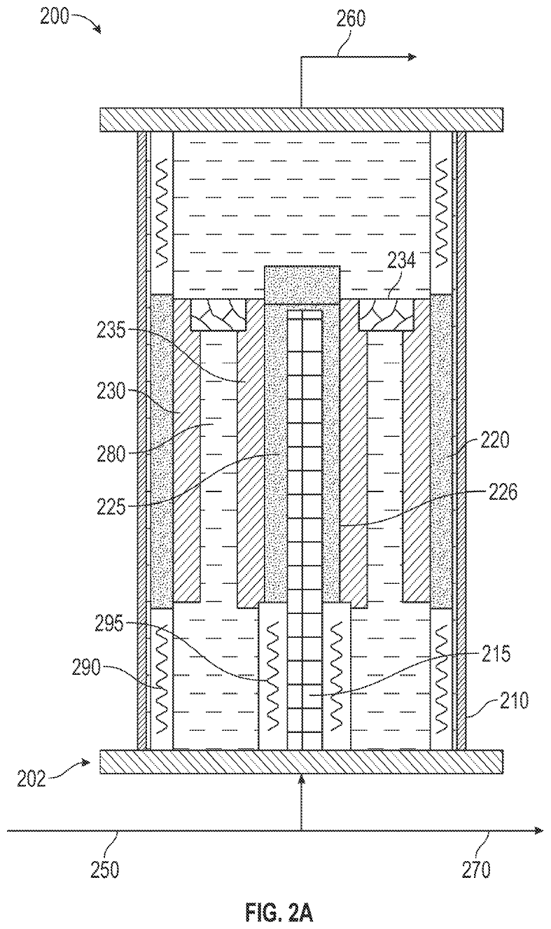

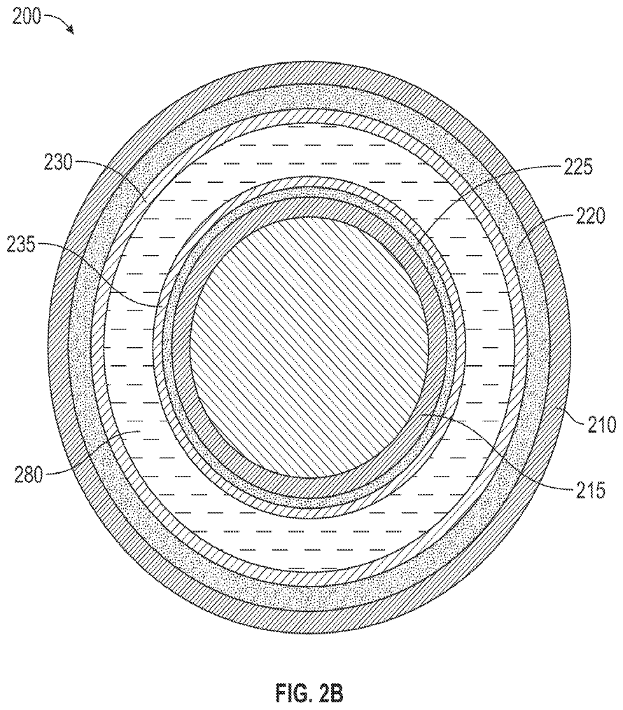

FIG. 2A is a cross section of an exemplary reaction vessel 200. The vessel may be similar to the vessel 100 portrayed in FIG. 1A in many respects. For example, as shown in FIG. 2A, the vessel 200 can be cylindrical and includes a grounded surface or electrode 210, a dielectric insulator 220, an internal high voltage electrode 230, a power supply/controller (as seen in FIG. 4), a gas inlet 250, a gas outlet 260, and a liquid outlet 270 as described above in connection with the vessel as portrayed in FIG. 1A.

As shown in FIG. 2A, the vessel 200 includes a first grounded surface or electrode 210, a first dielectric insulator 220, and a first high voltage electrode 230. The first grounded surface 210, first dielectric insulator 220, and first high voltage electrode 230 may each be similar or identical to the grounded surface electrode 110, the dielectric insulator 120, and the high voltage electrode 130 described elsewhere herein respectively. The vessel 200 also includes a second grounded surface 215, a second dielectric insulator 225, and a second high voltage electrode 235 dispersed annularly within the gas space 280 of FIG. 2A.

As shown in FIG. 2A, the second grounded surface or electrode 215 is configured to extend in the upward direction along the vessel 200 centerline from the bottom 202 of the vessel 200. In some embodiments, the second grounded surface or electrode 215 may extend in another direction from another portion of the reaction vessel 200. In some embodiments, the second grounded surface or electrode 215 may be suspended within the reaction vessel 200. In some embodiments, the reaction vessel 200 may not include a second grounded surface or electrode 215.

As shown in FIG. 2A, the second dielectric insulator 225 is positioned within in an annular arrangement along the vessel centerline and is configured to contact the second grounded surface or electrode 215. In some embodiments, the second dielectric insulator 225 may cover the entire second grounded surface or electrode 215. In some embodiments, the second dielectric insulator 225 may only cover a portion of the second grounded surface or electrode 215. In some embodiments, the second dielectric insulator 225 may incorporate corrugated ends 295 to reduce voltage tracking along the inner surface of the vessel.

In some embodiments, the second dielectric insulator 225 acts as an electric insulator for the electric field generated within the reaction vessel 200. The second dielectric insulator 225 acts as a barrier between the second high voltage electrode 235 and the second grounded surface or electrode 215. Such embodiments may be advantageous to enabling, enhancing, and/or increasing propagation reactions of hydrocarbons.

In some embodiments, the second dielectric insulator 225 may be composed of various materials. In some embodiments, the second dielectric insulator 225 may be composed of quartz, glass, ceramics, polymers, or other suitable dielectrics. In some embodiments the second dielectric insulator 225 may be impregnated and/or coated with catalytic materials to promote reaction kinetics. In some embodiments, the catalytic materials may comprise iron, magnesium, nickel, cobalt, thorium, ruthenium, or other suitable catalytic material. In some embodiments, the catalytic materials may comprise a mixture or combination of the materials described above or other suitable materials.

As shown in FIG. 2A, the second high voltage electrode 235 is suspended within the annulus of the reaction vessel 200. The second high voltage electrode 235 contacts the outer surface 226 of the second dielectric insulator 225. The second high voltage electrode 235 is also in contact with the free gas space, feedstock, reaction products, and/or any combination thereof. In some embodiments, the second high voltage electrode 235 is suspended within the dielectric annulus above the corrugated ends 295 of the second dielectric insulator 225 to minimize arcing potential to the second grounded surface or electrode 215.

The first and second high voltage electrodes 230 and 235 may be similar to the high voltage electrodes described elsewhere herein. In some embodiments, the length of the first or second high voltage electrodes 230 and 235 may be the same as each other or may be different from each other. In some embodiments, the first and second high voltage electrodes 230 and 235 may be 229 mm long.

The reaction vessel 200, as seen in FIG. 2A, includes a conductor 234 that contacts both the first and second high voltage electrodes 230 and 235. In some embodiments the conductor 234 may have multiple points of contact along the first and second high voltage electrodes 230 and 235. In some embodiments, the conductor 234 may be composed of copper wool or another suitable conductive material. The conductor 234 may be made from materials that are catalytic in nature to the desired reaction or impregnate with catalysts to promote reaction kinetics. This arrangement can advantageously distribute that the voltage potential from a high voltage source (not shown) evenly to both the first and second high electrodes 230 and 235, to maintain the same potential on both the first and second high voltage electrodes 230 and 235.

This configuration of the reaction vessel 200 as portrayed in FIG. 2A advantageously increases the ratio of the energized surface area of the dielectric insulators to the volume of free gas, (also referred to as the surface area to volume ratio), without reduction reaction effectiveness or efficiency. In some embodiments the size of the second grounded surface 215, the second dielectric surface 225, and the second high voltage electrode 235 may be increased to further increase the energized surface area to volume ratio. Additionally, this configuration of the reaction vessel 200 is advantageous to reducing gas arc failures by eliminating the voltage potential across the free gas space 280.

FIG. 2B depicts a top view of the annular reaction vessel 200 of FIG. 2A with a top portion of the reaction vessel 200 removed for ease of illustration. FIG. 2B shows the reaction vessel 200 and components configured in an annular arrangement. While an annular reaction vessel can advantageously be employed, other reaction vessel configurations can also be employed in certain embodiments. These configurations may include, but are not limited to: concentric electrodes configurations, multiple concentric electrodes configurations, parallel plate systems, multiple plate configurations, honeycomb configurations, cylindrical, spherical, or any other desired configuration. One of skill in the art would understand, aided by the current disclosure, that different vessel sizes and shapes can be used without departing from the scope of this invention.

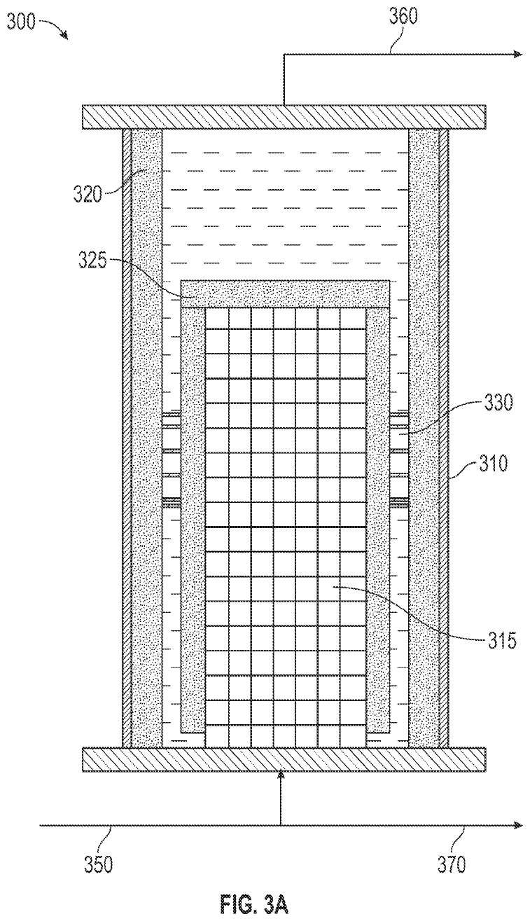

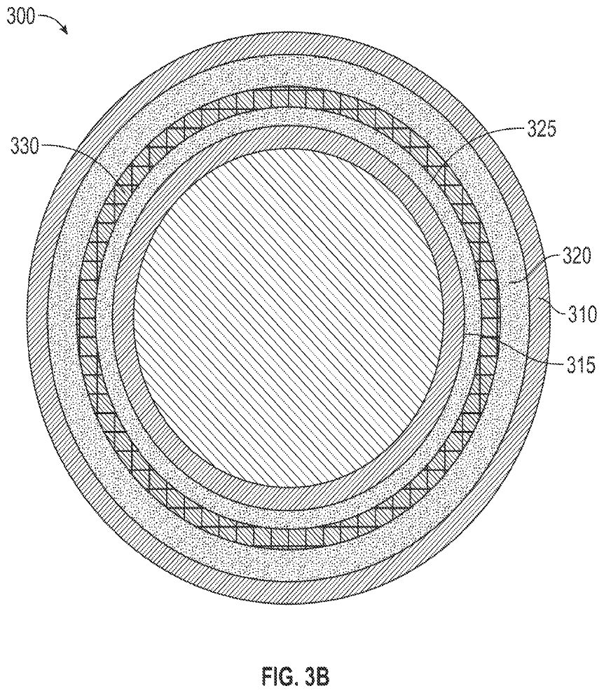

FIG. 3A is a cross section of an exemplary reaction vessel 300. The vessel 300 and components of the reaction vessel 300 may be similar to those vessels 100 and 200 and components of vessels 100 and 200 described elsewhere herein. For example, as shown in FIG. 3A, the vessel 300 includes a grounded surface or electrode 310, a dielectric insulator 320, an internal high voltage electrode 330, a power supply/controller, a gas inlet 350, a gas outlet 360, and a liquid outlet 370 similar to those described above.

As shown in FIG. 3A, the vessel 300 includes a first and second grounded surfaces 310 and 315 and a first and second dielectric insulators 320 and 325 which can be similar or identical to the grounded surface or electrode 110 and dielectric insulator 120 of vessel 100 or to the first and second grounded surfaces or electrodes 210 and 215 and first and second dielectric insulators 220 and 225 of vessel 200. In some embodiments, the central or concentric ground surface or electrode 315 can occupy a substantial volume of the reaction vessel.

The reaction vessel 300 in FIG. 3A includes a high voltage electrode 330. The high voltage electrode 330 contacts both the first and second dielectric insulators 320 and 325. The high voltage electrode 330 is disposed between and contacts the first and second dielectric surfaces 320 and 325. The high voltage electrode 330 can be disposed nearer one end of the second dielectric surface 325. In some embodiments, the high voltage electrode 330 can be positioned to contact the first and second dielectric insulators 320 and 325 at a point which is equidistant from the ends of the first dielectric insulator 320, the second dielectric insulator 325, or both. This configuration can advantageously reduce, minimize, or prevent uneven polarization of the dielectric surfaces 320 and 325. This configuration also advantageously reduces the amount of material needed to form the high voltage electrode 330 and potentially reduces the energy demands for the high voltage electrode 330, while maintaining adequate or sufficient charge on the surface of the dielectric insulators 320 and 325. In some embodiments, the high voltage electrode 330 does not need to completely cover the first and second dielectric insulator surfaces 320 and 325 for energy to be effectively dispersed across the first and second dielectric surfaces 320 and 325.

The high voltage electrode 330 can be similar or identical to the high voltage electrode 130 of vessel 100 or the first and second electrodes 230 and 235 of vessel 200. In some embodiments the length of the high voltage electrode 330 may be varied to reduce costs and materials. In some embodiments, the length of the high voltage electrode 330 may be 25.4 mm. This configuration may be advantageous in reducing the total mass of the high voltage electrode 330.

FIG. 3B depicts a top view of the reaction vessel 300 of FIG. 3A with a top portion removed for ease of illustration. FIG. 3B shows the reaction vessel 300 and components configured in an annular arrangement. While an annular reaction vessel can advantageously be employed, other reaction vessel configurations can also be employed in certain embodiments. These configurations may include, but are not limited to: concentric electrodes configurations, multiple concentric electrodes configurations, parallel plate systems, multiple plate configurations, honeycomb configurations, cylindrical, spherical, or any other desired configuration. One of skill in the art would understand, aided by the current disclosure, that different vessel sizes and shapes can be used without departing from the scope of this invention.

FIG. 4 is a cross section of an exemplary reaction vessel 400 with the components in a parallel plate arrangement. The vessel 400 may be similar or identical to the vessel 100, 200, or 300 portrayed in FIGS. 1A, 2A and 3A in many respects. For example, as shown in FIG. 4, the vessel 400 includes a grounded surface or electrode 410, a power supply/controller 440, a gas inlet 450, a gas outlet 460, and a liquid outlet 470 as described above in connection with the vessels 100, 200, and 300 portrayed in FIGS. 1A, 2A, and 3A. Additionally, reaction vessel 400 includes a plurality of internal grounded surfaces or electrodes 415, a plurality of high voltage electrodes 430, and a plurality of dielectric insulators 420.

The grounded surface or electrode 410 is the reaction vessel wall 414. The plurality of internal grounded surfaces or electrodes 415 are positioned within the free gas space 480 of reaction vessel 400. In some embodiments, the plurality of internal grounded surfaces or electrodes 415 may be configured such that at least one of the plurality of internal grounded surfaces or electrodes 415 is in contact with the reaction vessel wall 414. In some embodiments, multiple grounded surfaces or electrodes 415 may contact the reaction vessel wall 414. In some embodiments, a separate electrical connection to the plurality of grounded surfaces or electrode 415 may maintain the plurality of internal grounded surfaces 2 or electrodes 415 at ground. In some embodiments, the plurality of grounded surfaces or electrodes 415 comprises 2, 3, 4, or 5, or any other number of grounded surfaces or electrodes.

Vessel 400 includes a plurality of dielectric insulators 420. The plurality of dielectric insulators 420 are positioned in the free gas space 480 of the reaction vessel 400. In some embodiments, at least one of the plurality of dielectric insulators 420 may be configured to contact the reaction vessel wall 414. In some embodiments, more than one of the plurality of dielectric insulators 420 may be configured to contact the reaction vessel wall 414.

As shown in FIG. 4, at least one of the plurality of dielectric insulators 420 contacts at least one of the plurality of the internal grounded surfaces or electrodes 415. In some embodiments, the plurality of dielectric insulators 420 may cover the plurality of the internal grounded surfaces or electrodes 415 and the reaction vessel wall 414. In some embodiments, the plurality of dielectric insulators 420 may only cover a portion of the plurality of internal grounded surfaces or electrodes 415 and the reaction vessel wall 414. In some embodiments, the plurality of dielectric insulators 420 may incorporate corrugated ends to reduce voltage tracking along the inner surface 412 of the vessel 400.

In some embodiments, the plurality of dielectric insulators 420 act as electric insulators for the electric field. In some embodiments, the plurality of dielectric insulators 420 can act as a barrier between the plurality of high voltage electrodes 430 and the plurality of internal grounded surfaces or electrodes 415 and the grounded surface or electrode 410. Such embodiments may be advantageous to enabling, enhancing, and/or increasing propagation reactions of hydrocarbons.

In some embodiments, the plurality of dielectric insulators 420 may be composed of various materials. In some embodiments, the plurality of dielectric insulators 420 may be composed of quartz, glass, ceramics, polymers, or other suitable dielectrics. In some embodiments the plurality of dielectric insulators 420 may be impregnated and/or coated with catalytic materials to promote reaction kinetics. In some embodiments, the catalytic materials may comprise iron, magnesium, nickel, cobalt, thorium, ruthenium, or other suitable catalytic material. In some embodiments, the catalytic materials may comprise a mixture or combination of the materials described above or other suitable materials. In some embodiments, the plurality of dielectric insulators 420 may be formed of one or more catalytic materials.

As shown in FIG. 4, the vessel 400 includes a plurality of high voltage electrodes 430. The plurality of high voltage electrodes 430 are positioned in free gas space 480 of the reaction vessel 400. Each of the plurality of high voltage electrodes 430 is configured proximate to or in contact with at least one of the plurality of dielectric insulators 420. In some embodiments, each of the plurality of high voltage electrodes 430 is located proximate two dielectric insulators of the plurality of dielectric insulators 420, with one dielectric insulator 420 positioned on each side of the high voltage electrode 430. In some embodiments, each of the plurality of high voltage electrodes 430 is configured to contact at least one of the plurality of dielectric insulators 420. In some embodiments, each of the plurality of high voltage electrodes 430 is configured to contact at least two of the plurality of dielectric insulators 420.

The plurality of high voltage electrodes 430 may be similar or identical to the high voltage electrode 130 of vessel 100, the first and second high voltage electrodes 230 and 235 of vessel 200, or the high voltage electrodes 330 of vessel 300 described elsewhere herein. In some embodiments, the plurality of high voltage electrodes 430 may be suspended between the corrugated ends of the plurality of dielectric insulators 420 to minimize arcing potential to the plurality of internal grounded surfaces or electrodes 410.

In some embodiments the length of each of the plurality of high voltage electrodes 430 is less than the length of the dielectric insulator 420 proximate which the high voltage electrode 430 is located. Where the high voltage electrode 430 extends less than the entire length of a corresponding dielectric insulator 420, the high voltage electrode 430 can generate a uniform electric field over the whole or a substantial portion of the surface of the dielectric electrode 420 proximate which the high voltage electrode 430 is located.

The reaction vessel 400 includes a power supply/controller 440. The power supply/controller 440 is configured to supply power to the plurality of high voltage electrodes 430 to generate an electric field. In some embodiments, the power supply/controller 440 can be coupled to the high voltage electrodes 430, for example, via a high voltage cable 442. In some embodiments, a high voltage cable 442 can extend between the power supply/controller 440 and the electrodes 430 through the gas inlet 450. In some embodiments, the high voltage cable 442 can extend between the power/supply controller 440 and the high voltage electrode 430 through the gas outlet 460. In some embodiments, the vessel 400 can include a high voltage entrance device 444. The high voltage entrance device 444 may be a high voltage insulator entrance bushing.

In some embodiments, power can be supplied to the electrodes 430 via conductors (not shown) as an application voltage of between 100 VAC and 100,000 VAC, between 100 VAC and 10,000 VAC, between 500 VAC and 1,500 VAC, about 500 VAC, about 1000 VAC, about 5,000 VAC, about 10,000 VAC, about 45,000 VAC, about 50,000 VAC, about 60,000 VAC or any other suitable application voltage. In some embodiments, the voltage range can advantageously be between 12,000 VAC and 15,000 VAC, or between 35,000 VAC and 60,000 VAC,

In certain embodiments, power can be supplied to the electrodes 430 in the form of an application frequency of between 60 hertz to 10,000 hertz, or any other suitable frequency. In some embodiments, the frequency can advantageously be between 1,000 hertz and 1,200 hertz.

As shown in FIG. 4, the components of the vessel 400 are configured as a parallel plate system. In some embodiments, the components of the vessel 400 may be configured in multiple plate configuration. In some embodiments, a multiple plate configuration can include two or more plates positioned parallel to an axial center line of a vessel. One of skill in the art would understand, aided by the current disclosure, that different number of plates could be used without departing from the scope of this invention. FIG. 4 depicts a vessel 400 having 4 high voltage electrodes 430. A vessel having more or fewer than 4 high voltage electrode and dielectric portions does not depart from the scope of this disclosure.

While a parallel plate system can advantageously be employed, other reaction vessel configurations can also be employed in certain embodiments. These configurations may include, but are not limited to: concentric electrodes configurations, multiple concentric electrodes configurations, annular component configurations, multiple plate configurations, honeycomb configurations, cylindrical, spherical, or any other desired configuration. One of skill in the art would understand, aided by the current disclosure, that different vessel sizes and shapes can be used without departing from the scope of this invention.

The reaction vessel 400 as shown in FIG. 4 is advantageously configured to increase the energized surface area of the dielectric insulators 420 while simultaneously reducing the volume of the free gas 480. This increase in energized surface area to volume ratio led to an increase in reaction effectiveness.

Tests were conducted using vessels having the configuration of the vessels as show in FIGS. 1A-B, 2A-B, 3A-B. In all runs, the feed stream was composed of compressed natural gas (CNG). As the CNG reacted and propagated within the vessel, various components, including liquid petroleum gas (LPG) was formed and was measured in the product stream. As described herein, LPG refers to a combination of propane, isobutane, butane, and propylene. In some embodiments, LPG may also comprise butylene. The energized surface area of the dielectric insulators and the free gas volume arrangement of the reaction vessel were varied for each run to determine the effect the energized surface area to volume ratio has on reaction effectiveness. The results of the tests are shown in TABLE I.

TABLE 1

| | | | | | | | | | Surface | | | | Increase |
|---|---|---|---|---|---|---|---|---|---|---|---|---|---|
| | Vessel ID | Outer Dielectric | | Electrode | Inner Dielectric | | Gas | Inner | Area | Free | [symbol] /vol | Peak | ( [symbol] |
| | GRD | OD | ID | Length | OD | ID | Gap | GRD | (energized) | Gas Val | Ratio | [symbol] | base) |
| Run | (mm) | (mm) | (mm) | (mm) | (mm) | (mm) | (mm) | ( [symbol] ) | (mm2) | (mm [symbol] ) | (mm2/mm3) | (%) | (%) |
| 1 | 90 | 85 | 80 | 229 | 0 | 0 | 40* | 0 | [symbol] | [symbol] | 0.05 | 0.55 | |
| 2 | 90 | 85 | 80 | 229 | 42 | [symbol] | 19 | [symbol] | [symbol] | [symbol] | [symbol] | 1.42 | 258% |
| 3 | 90 | 85 | 80 | 229 | 64 | 60 | 8 | 60 | [symbol] | [symbol] | [symbol] | 1.74 | [symbol] |
| 4 | 90 | 85 | 80 | [symbol] | [symbol] | 60 | 8 | 0 | [symbol] | [symbol] | [symbol] | [symbol] | −35% |
| 5 | 90 | 85 | 80 | 25.4 | 75 | 70 | 2.5 | 70 | [symbol] | [symbol] | 0.800 | [symbol] | 431% |

Note:
Run 4 increase is not compared to Run 1, but to Run 2
Note:
The ground reference reduction in Run 4 is approximately equal to the conversion reduction from Run 2. [symbol]
indicates data missing or illegible when filed

Run 1 was conducted using the reactor vessel 100 of FIGS. 1A-B. Run 2 was conducted using the reactor vessel 200 of FIGS. 2A-B. Run 3 was conducted using the reactor vessel 200 of FIGS. 2A-B, where the diameters of the inner dielectric insulator 225 and the inner grounded surface or electrode 215 were increased. Run 4 was conducted using the reactor vessel 200 of FIGS. 2A-B, where the inner grounded surface or electrode 215 was not present. Run 5 was conducted using the reactor vessel 300 of FIGS. 3A-B.

All runs were conducted at a pressure of approximately 2 psig and at a voltage that ranged from 35,000 VAC to 50,000 VAC. The dielectric insulator used in each run was composed of quartz.

The retention time of the free gas was held constant across all runs. The retention time of the free gas within the reaction vessel may be altered to produce different hydrocarbon compositions in the product stream. For example, a longer retention time results in more propagation reactions and a higher concentration of longer hydrocarbon chains in the product stream.

As described above, the feed stream in the runs was composed of CNG. In some embodiments, the composition of the gaseous feed stream may be altered to comprise a pure hydrocarbon or a mixture of hydrocarbons according to the desired reaction product.

The desired reaction product in runs 1-5 was LPG. The data in Table 1 provide a peak LPG, which indicates the percentage of the product stream comprising LPG. The Increase (to base) value shows how a subsequent run's peak LPG changed relative to a prior run. The peak LPG data show that as the energized surface area to volume ratio increased the percentage of LPG in the product streams generally increased, except in run 4, where peak LPG reduced. As noted above, Run 4 was conducted using the reactor vessel of FIGS. 2A-B, where the inner grounded surface or electrode was not present. The absence of the inner grounded surface or electrode resulted in the inner dielectric insulator failing to become completely polarized or relaxed during the applied voltage cyce. This, in turn, led to a difference in voltages between the inner and outer dielectric insulators which caused arcing to occur between the two dielecrics. As a result, the uniformity of the charge along the dielectric surfaces was decreased, thus reducing the propagation reactions that were occuring within the vessel. For these reasons, peak LPG was reduced for Run 4. The data in Table 1 demonstrate that as the energized surface area to volume ratio increases, the desirable production of LPG increases. Additionally, as the energized surface area to volume ratio increases, the material requirements and energy requirements can also advantageously be reduced. The energized surface area to volume results indicate that the propagation reactions in the reaction vessels described herein occur on or near the surface of the dielectric insulators, and occur to a lesser extent, in the bulk volume of the gas and the reaction vessel.

As a prophetic example, CNG is introduced into a reaction vessel with 28 plates arranged parallel to each other, similar to the reaction vessel 400 of FIG. 4. The arrangement has an energized surface area to volume ratio of about 1.97. The pressure is held constant at 2 psig within the reaction vessel and the retention time of the free gas is similar to those described above. CNG is supplied as a feed gas, and LPG is measured as a desired product gas. The resulting percentage of LPG within the product stream is about 3.89%, which is an increase of about 700% from Run 1 shown and a 164% increase over Run 5 in Table 1 above.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component or directly connected to the second component. As used herein, the term “plurality” denotes two or more. For example, a plurality of components indicates two or more components.

In the foregoing description, specific details are given to provide a thorough understanding of the examples. However, it will be understood by one of ordinary skill in the art that the examples may be practiced without these specific details.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. A reaction vessel comprising:
a vessel wall forming an internal volume comprising a fluid space;
at least one grounded surface or electrode;
at least one dielectric insulator, the at least one dielectric insulator in contact with the at least one grounded surface or electrode; and
at least one high voltage electrode disposed within the reaction vessel, wherein a first portion of the at least one high voltage electrode is in contact with the at least one dielectric insulator, and wherein a second portion of the at least one high voltage electrode is in contact with the fluid space.

2. The reaction vessel of claim 1 wherein the vessel wall comprises the at least one grounded surface or electrode.

3. The reaction vessel of claim 1, wherein the at least one grounded surface or electrode is formed on the vessel wall.

4. The reaction vessel of claim 1, wherein the at least on high voltage electrode is configured to generate an electric field within the reaction vessel.

5. The device of claim 1, wherein the reaction vessel further comprises a power supplier electrically connected to the at least one high voltage electrode, the power supplier configured to supply power to the at least one high voltage electrode in a form of an application voltage between 30,000 VAC and 50,000 VAC.

6. The reaction vessel of claim 1, wherein the at least one dielectric insulator is disposed on an inner surface of the at least one grounded surface or electrode.

7. The reaction vessel of claim 1, wherein the reaction vessel is cylindrical.

8. The reaction vessel of claim 7, wherein the at least one high voltage electrode and the at least one dielectric insulator are disposed concentrically or coaxially within the reaction vessel.

9. The reaction vessel of claim 1, wherein the reaction vessel is a rectangular prism and wherein the at least one high voltage electrode is a plate electrode.

10. The reaction vessel of claim 9, comprising a plurality of high voltage electrodes and a plurality of dielectric insulators arranged parallel to each other within the reaction vessel.

11. The reaction vessel of claim 1, wherein the energized surface area to volume ratio is between 0 and 1.

12. The reaction vessel of claim 1, wherein the at least one high voltage electrode comprises a mesh.

13. The reaction vessel of claim 12, wherein the at least one high voltage electrode is configured to allow a fluid to flow through the mesh of the at least one high voltage electrode.

14. A method for propagating hydrogens, the method comprising:
providing a feed fluid to a reaction vessel via a fluid inlet, wherein the reaction vessel comprises:
a grounded surface or electrode;
a dielectric insulator in contact with the grounded surface or electrode;
a high voltage electrode in contact with the dielectric insulator and a fluid space of the reaction vessel;
generating an electric field within the fluid space of the reaction vessel;
reacting one or more components of the feed fluid in the electric field; and
extracting one or more components of interest from the reaction vessel, via a fluid outlet of the reaction vessel.

15. The method of claim 14, wherein generating an electric field comprises creating a plasma in the fluid space near the surface of the high voltage electrode.

16. The method of claim 15, wherein the high voltage electrode at between 30,000 VAC and 50,000 VAC.

17. The method of claim 14, further comprising adjusting a retention time of the feed fluid within the reaction vessel such that feed fluid remains in the reaction vessel for between 30 seconds and 5 minutes.

18. The method of claim 14, wherein the feed fluid is compressed natural gas (CNG).

19. The method of claim 14, wherein the feed fluid comprises one or more hydrocarbon components, and wherein reacting the one or more hydrocarbon components of the feed fluid comprises propagating the hydrocarbon components into larger hydrocarbon chains.

20. The method of claim 19, wherein extracting the component of interest comprises extracting hydrocarbon chains from the reaction vessel.
